(19) United States
(12) Patent Application Publication
(10) Pub. No.: US 2006/0259046 A1
(43) Pub. Date: Nov. 16, 2006

(54) Body tissue incision closing instrument

(75) Inventors: Roger A. de la Torre, Wentzville, MO (US); Matthew P. LaConte, Maryland Heights, MO (US)

Correspondence Address: THOMPSON COBURN, LLP, ONE US BANK PLAZA, SUITE 3500, ST LOUIS, MO, 63101, US

(73) Assignee: TowerTech Research Group

(21) Appl. No.: 11/128,483

(22) Filed: May 13, 2005

(51) Int. Cl. A61B 17/04

(52) U.S. Cl. 606/144

(57) ABSTRACT

A surgical instrument that is employed in closing a surgical incision made deep into body tissue holds a length of suture and a pair of needles secured to the opposite ends of the suture. The instrument is configured for insertion of the instrument into a body tissue incision, for deploying the needles and suture through body tissue on opposite sides of the incision, and for then withdrawing the needles and suture ends from the incision to tie a knot in the suture ends and thereby close the incision.

### BACKGROUND OF THE INVENTION

(1) Field of the Invention

The present invention pertains to a surgical instrument that is employed in closing a surgical incision made deep inside body tissue, for example an incision made in a laparoscopic surgical procedure. More specifically, the present invention pertains to a surgical instrument that holds a length of suture and a pair of needles that are secured to the opposite ends of the suture. The instrument is configured for insertion of the instrument into a body tissue incision, for deploying the needles and suture through the body tissue on opposite sides of the incision, and for then withdrawing the needles and suture ends from the incision to tie a knot in the suture ends and thereby close the incision.

(2) Description of the Related Art

In laparoscopic surgical procedures, a small incision is typically made through body tissue to access a surgical site inside the body, and a trocar is typically inserted through the incision. With the trocar in place, the body cavity accessed by the trocar can be insufflated to provide ample room at the surgical site for the manipulation of surgical instruments. The surgical instruments access the surgical site by being inserted through the interior bore of the trocar.

When the surgery is completed, the trocar is withdrawn. Incisions made in the trocar that are less than 10 mm in diameter will heal without complication if not closed internally, i.e. deep inside the incision. However, trocars with diameters greater than 10 mm are commonly used, and therefore the incisions made in the body tissue for insertion of these trocars must be closed. As a result, there is a need to close these larger incisions made for the larger diameter trocars to prevent the contents of the abdominal cavity from herniating into the incision following the surgery.

### SUMMARY OF THE INVENTION

The present invention is an incision closing instrument that closes deep body tissue incisions made for the insertion of a trocar. The instrument and its method of use provide a means of effectively closing deep tissue incisions to prevent trocar site herination of the tissue. The instrument and its method of use enable a consistent and fast placement of suture across the incision deep inside body tissue by a surgeon manipulating the instrument from outside the incision.

The body tissue incision closing instrument of the invention is comprised of a handle having an elongate length with opposite proximal and distal ends. The distal end of the handle is configured for insertion deep into a body tissue incision. The proximal end of the handle is configured for easy manipulation by the hand of the surgeon.

A pair of actuators extend along the length of the handle. Each of the actuators is mounted to the handle for reciprocating movement of the actuator along the handle length. Each actuator has a trigger that is positioned at a side of the handle adjacent the handle proximal end. The positions of the triggers are easily accessible by a finger of the surgeon's hand manipulating the proximal end of the handle.

The ends of the actuators opposite the triggers extend from the handle distal end and are connected to needle holders. Each of the needle holders is configured for removably holding a needle. The needle holders are positioned side-by-side and are spaced from the handle distal end. Manipulating the actuator triggers selectively moves each of the needle holders toward and away from the handle distal end.

A pair of needles is removably connected to the pair of needle holders. The needles are held on the needle holders with the points or tips of the needles directed toward the handle distal end. The pair of needles are secured to a single length of suture material, with each needle being secured to one of the opposite ends of the suture material.

A pair of needle receivers is provided on the handle distal end. The pair of needle receivers oppose the pair of needles with a spacing between the needle receivers and the needle points. The needle receivers are configured to grasp the needles firmly when the needle points are received by the needle receivers, enabling the needle receivers to separate the needles from the needle holders.

In use of the body tissue incision closing instrument, the proximal end of the instrument handle is held and maneuvered by the surgeon's hand. The handle distal end is inserted into a tissue incision and is positioned at a surgical site where the incision is desired to be closed. The handle is manipulated so that a portion of the body tissue at one side of the incision is positioned in the space between one of the needle points and the opposite needle receiver. When so positioned, the surgeon then pulls on the actuator trigger operatively connected to the positioned needle, causing the needle point to move through the tissue and into the needle receiver. This causes the needle receiver to firmly grasp the needle. Releasing the trigger then causes the needle holder to move back to its original position, causing the needle receiver to remove the needle from the needle holder.

The instrument is then turned so that a portion of the tissue on the opposite side of the incision is positioned between the remaining needle point and its associated needle receiver. The trigger for this needle is then operated, causing the needle holder to move toward the handle distal end, and thereby causing the needle point to pass through the tissue and be received in the needle receiver. The trigger is then again released, causing the needle holder to move back to its original position and causing the needle receiver to separate the needle from the needle holder.

Withdrawing the instrument from the incision with the two needles held by the two needle receivers causes the length of suture attached to the two needles to be pulled through the needle holes at the opposite sides of the incision. Removing the instrument from the incision provides access to the opposite ends of the suture held by the needle receivers. Tying and tightening a knot in the suture ends then closes the incision.

Thus, the body tissue incision closing instrument provides a quick and effective means for closing incisions deep in body tissue.

### BRIEF DESCRIPTION OF THE DRAWINGS

Further objects and features of the invention are set forth in the following detailed description of the preferred embodiment of the invention and in the following drawing figures wherein:

FIG. 1 is a perspective view of the body tissue incision closing instrument of the invention;

FIG. 2 is an enlarged partial view of the distal end of the instrument shown in FIG. 1;

FIG. 3 is a partial view of the instrument distal end in an initial step of using the instrument;

FIG. 4 is a view similar to FIG. 3, but showing a subsequent step in the use of the instrument;

FIG. 5 shows a step of using the instrument subsequent to that of FIG. 4;

FIG. 6 shows a step of using the instrument subsequent to that of FIG. 5;

FIG. 7 shows a step of using the instrument subsequent to that of FIG. 6; and,

FIG. 8 shows a step of using the instrument subsequent to that of FIG. 7.

### DETAILED DESCRIPTION OF THE PREFERRED EMBODIMENT

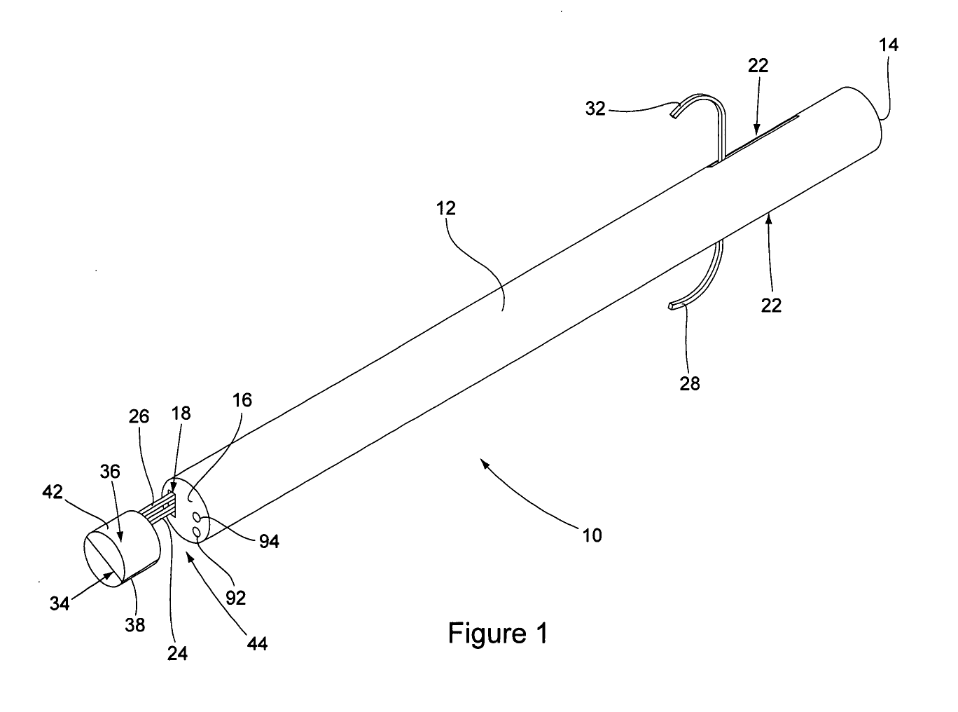

The body tissue closing instrument of the invention 10 is shown in FIG. 1. The component parts of the instrument to be described are all constructed of materials typically used in surgical instruments. The instrument is constructed with an elongate handle 12 having opposite proximal 14 and distal 16 ends. In the preferred embodiment, the handle 12 is narrow and has a smooth cylindrical exterior surface to facilitate movement of the handle through the interior of a trocar, or movement of the handle in a body tissue incision. Although the handle 12 is shown as having a straight and narrow configuration in the drawings, the handle could be curved or be given other configurations that would facilitate its use. In addition, the handle proximal end 14 could be given other configurations that comfortably fit a person's hand for easily manipulating the handle.

The handle has an interior bore 18 that extends through a majority of the handle length. The bore 18 emerges from the handle distal end 16 as shown in FIG. 1. As the bore 18 extends from the handle distal end 16 toward the handle proximal end 14, the bore splits and angles toward the diametrically opposite sides of the handle. The split branches of the bore emerge from the opposite sides of the handle 12 as a pair of slots 22 that are positioned adjacent the handle proximal end 14.

A pair of actuators in the form of elongate, narrow rods 24, 26 extend through the handle bore 18. The exterior dimensions of the rods 24, 26, and the interior dimension of the handle bore 18 enable the pair of rods to be easily reciprocated along the handle length. Proximal ends of the rods 24, 26 emerge from the bore through the handle slots 22 and are formed as triggers 28, 32. The slots 22 position the triggers 28, 32 adjacent the handle proximal end 14 where the triggers are easily accessible by a finger of a person's hand manipulating the handle proximal end. A pair of springs (not shown) are provided on the rods 24, 26 inside the handle bore 18. The springs bias the rods 24, 26 and their triggers 28, 32 away from the handle proximal end 14 and toward the handle distal end 16.

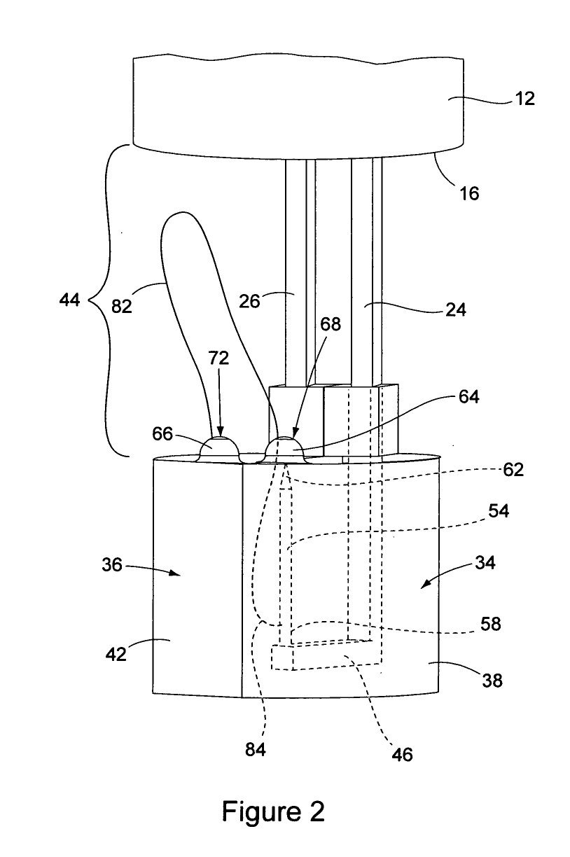

The opposite distal ends of the rods 24, 26 project from the handle distal end 16 and are connected to a pair of needle holders 34, 36. The needle holders 34, 36 are basically mirror images of each other and both function in the same manner. Therefore, only one of the needle holders 34, and its functioning, will be described in detail. Each of the needle holders 34, 36 has an exterior housing 38, 42 that has the configuration of a half cylinder. As shown in FIGS. 1 and 2, the housings 38, 42 are positioned side-by-side so that together they have a cylindrical configuration. The cylindrical configurations of the housings 38, 42 is dimensioned to match the cylindrical configuration of the instrument handle 12. This gives the instrument 10 a smooth, narrow exterior configuration that can be easily moved through a trocar and easily moved in a tissue incision. Each of the housings 38, 42 is mounted to the distal ends of the respective actuator rods 24, 26 for reciprocating movement of the housings over the rod distal ends. A spring mechanism (not shown) is provided in each housing 38, 42. The spring mechanisms in the housings 38, 42 bias the housings to first positions of the housings relative to the distal ends of the actuator rods 24, 26 as shown in FIG. 2. In the first positions of the housings 38, 42 on the ends of their respective actuator rods 24, 26, there is a spacing 44 between the housings 38, 42 and the rod distal end 16 that is sufficiently large to position a portion of body tissue in the spacing, as will be explained.

Each of the needle holder housings 38, 42 contains a mechanism 46 for removably holding a needle. One mechanism 46 is shown schematically in FIG. 2. Each mechanism 46 is secured to a distal end of each of the actuator rods 24, 26 for movement with the actuator rods. Each mechanism 46 holds the needle end that is opposite the needle point. In addition, the mechanism 46 removably holds the needle end to which a length of suture is attached, and thereby removably holds the suture end secured to the needle.

A pair of needles 52, 54 is removably connected to the needle holding mechanisms contained in each needle holder housing 34, 36. Only one of the needles 54 is shown in the schematic representation of FIG. 2. As shown, the needle 54 has a straight length with one end 58 removably held by the needle holding mechanism 46, and the opposite end 62 having a point. The needle held end 54 is removably held by the needle holder mechanism 46 in a manner that allows the needle 54 to be removed from the mechanism 46 by a slight pulling force exerted on the needle 54. This connection can be achieved by a friction fit between the needle holding mechanism 46 and the needle end 58. As shown in FIG. 2, the needle holding mechanism 46 holds the needle 54 with the needle point 62 directed toward the instrument distal end 16. The second needle 56 is held by an identical needle holding mechanism contained in the other needle holder housing 42.

With the needle holder housing 38 in its first position relative to the actuator rod 24 shown in FIG. 2, the housing 38 completely surrounds the needle 54 and the needle point 62. Each needle holder housing 38, 42 is provided with a protruding portion 64, 66 that is positioned adjacent the point of the needle contained in the housing. Each housing protruding portion 64, 66 has a needle aperture 68, 72 adjacent the needle point. The apertures 78, 72 are positioned adjacent the needle points so that the points and the needles will extend through the apertures on movement of the needle holder housings 38, 42 from their first positions shown in FIG. 2, toward second positions of the housings relative to their respective actuator rods 24, 26 that are explained later.

A single length of suture material 82 is connected to the pair of needles 54, 56. The suture material 82 is shown schematically in FIG. 2 to represent any type of suture material or suturing means that may be used with the needles. The single length of suture material 82 has opposite ends 84, 86 connected to the respective needles 54, 56. FIG. 2 shows one end 84 of the single length of suture material secured to one of the needles 54. As shown in FIG. 2, the suture end 84 is secured adjacent the needle end 58 held by the needle holding mechanism 46 of the needle holder 34. The end of the suture extends into the needle holder housing 38 to the end of the needle held by the mechanism 46, but the housing 38 does not obstruct or restrain the suture end. Thus, the suture end is free to exit the needle holder housing 38.

A pair of needle receivers 92, 94 is provided in the handle distal end 16. The needle receivers 92, 94 are positioned directly opposite the respective needles 54, 56 held in the needle holders 34, 36. Each of the needle receivers 92, 94 functions to grasp and firmly hold the respective opposite needle 54, 56 when the needle is inserted into the needle receiver. The grasping force of the needle receivers 92, 94 exerted on the needles 54, 56 inserted into the needle receivers is greater than the force of the needle holding mechanisms 46 in each of the needle holders 34, 36. Thus, the grasping force of the needle receivers 92, 94 is sufficient to remove the needles 54, 56 from the needle holding mechanisms contained in the needle holders 34, 36.

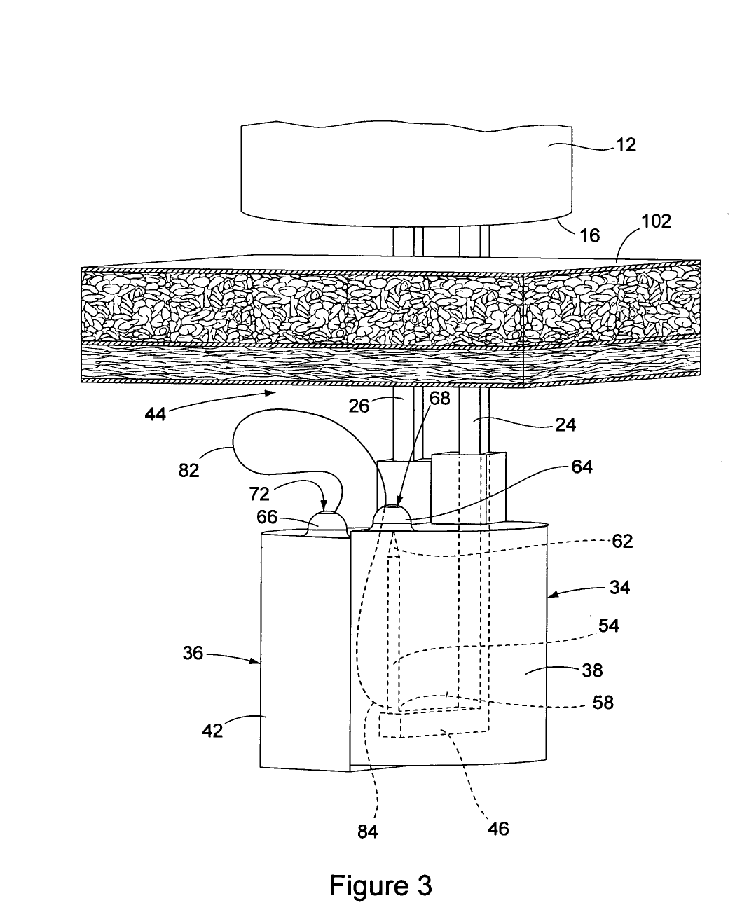

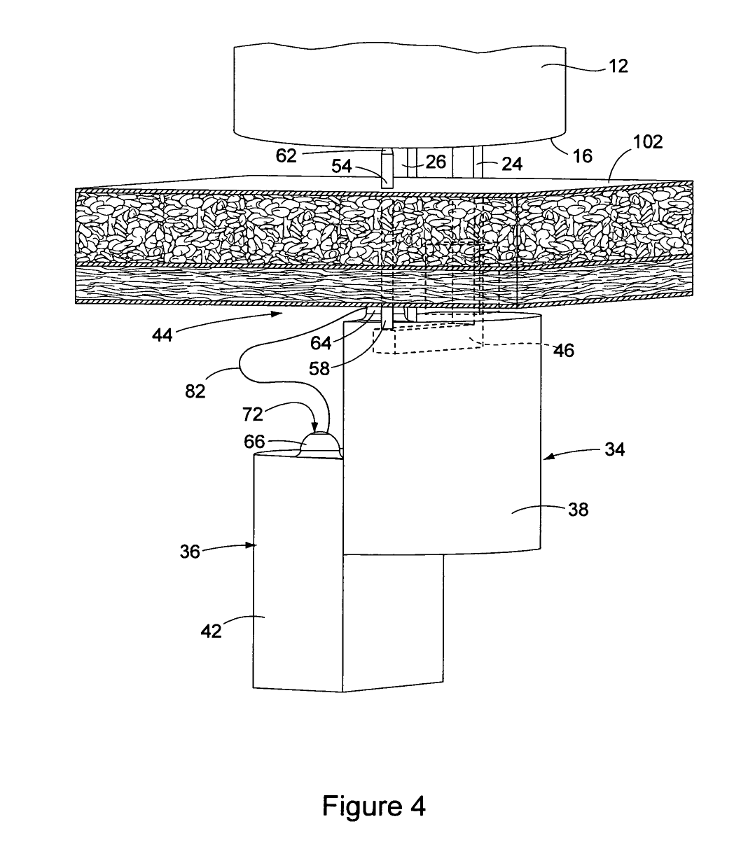

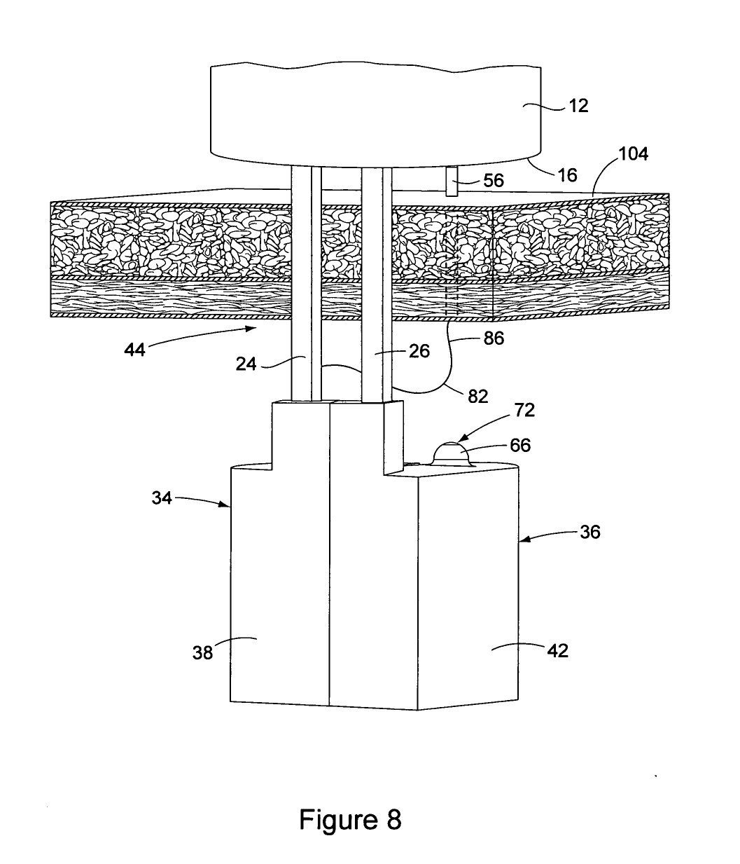

One example of using the body tissue incision closing instrument 10 is illustrated schematically in FIGS. 3-8. In use of the instrument, the proximal end 14 of the instrument handle 12 is held by the surgeon's hand and maneuvered to position the handle distal end 16 through a trocar or through an incision to the desired incision location to be closed. FIG. 3 illustrates the handle distal end 16 positioned with the distal ends of the actuator rods 24, 26 extending through an incision in body tissue. A portion of the body tissue 102 on one side of the incision is shown in front of the actuator rods 24, 26 in FIG. 3. A second portion of the body tissue on an opposite side of the incision is not shown in FIG. 3 to simplify the drawing figures, but would be positioned on the opposite side of the actuator rods 24, 26. The handle 12 is manipulated by the surgeon so that the first portion of the body tissue 102 at one side of the incision is positioned in the spacing 44 between the needle holders 34, 36 of the instrument and the instrument distal end 16. This also positions the first portion of the tissue 102 between the needle point 62 of the first needle holder 34 and the needle receiver 92 opposite the needle point. When so positioned, the surgeon then pulls on the actuator trigger 28 operatively connected to the positioned needle 54, causing the distal end of the actuator rod 24, the mechanism 46 removably holding the needle, the needle 54 and the needle holder housing 38 to all move upwardly as shown in FIG. 3. All of these component parts of the instrument will continue to move upwardly as the trigger 28 is pulled toward the instrument proximal end 14 until the needle holder housing 38 comes into contact with the underside of the tissue portion 102. This restricts further movement of the housing 38 upwardly. At this point, the needle holding mechanism 46 and the needle 54 will continue to move upwardly relative to the needle housing 38 as the trigger 28 is continued to be pulled toward the instrument proximal end 14. This causes the needle point 62 to move upwardly through the needle aperture 68 in the housing protruding portion 64 and enter into the tissue portion 102. The needle point 62 moves through the tissue portion 102 and begins to enter into the opposing needle receiver 92 as shown in FIG. 4. At the full extent of movement of the trigger 28 toward the handle proximal end 14, the needle 54 is pushed through the tissue portion 102 and is inserted into the opposing needle receiver 92. The needle receiver 92 firmly grasps the needle 54. In this position of the trigger 28 relative to the handle proximal end 14, the needle holder 36 has been moved to its second position relative to its actuator rod 24 and the instrument handle 12.

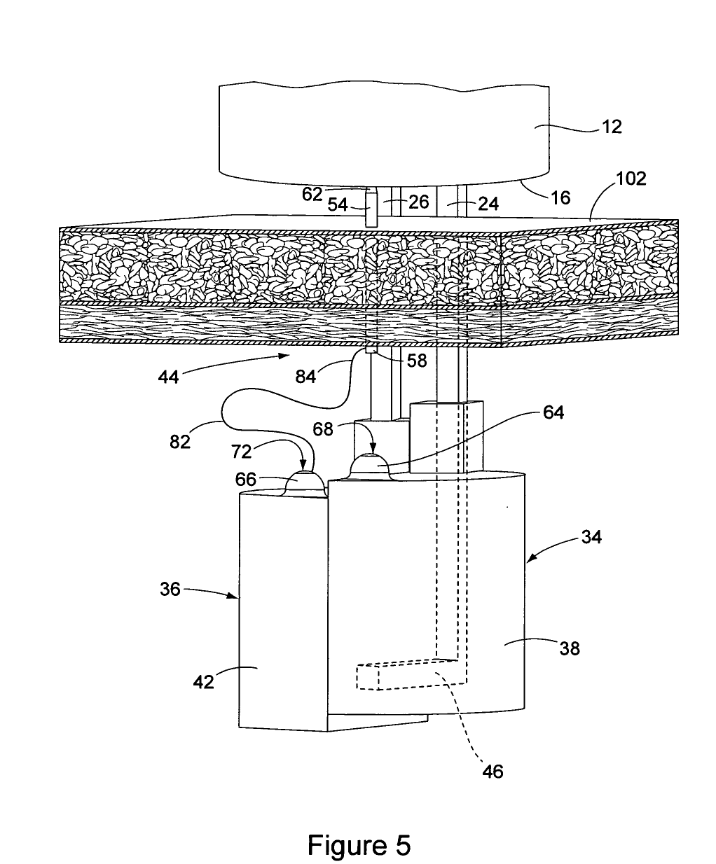

On subsequent release of the trigger 28, the spring (not shown) contained in the handle 12 biases the actuator rod 24 and the needle holder 34 toward their first positions relative to the handle 12 shown in FIGS. 1 and 2. This causes the needle receiver 92 to pull the needle 54 from the needle holding mechanism 46 in the needle holder 34. The needle 54 is securely held by the needle receiver 92 as the movement of the actuator rod 24 causes the needle holder 34 to move back toward its first position relative to the handle 12 as shown in FIG. 5. As the needle holder 34 is moved back toward its first position relative to the handle 12, the needle 54 securely held by the needle receiver 92 removes the end of the suture material 84 secured to the needle from the needle holder housing 38.

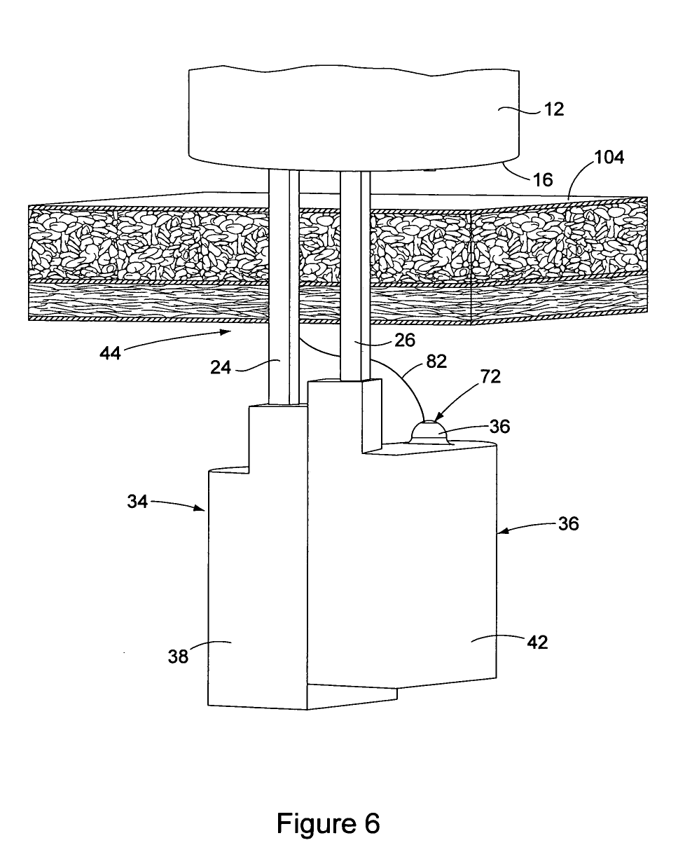

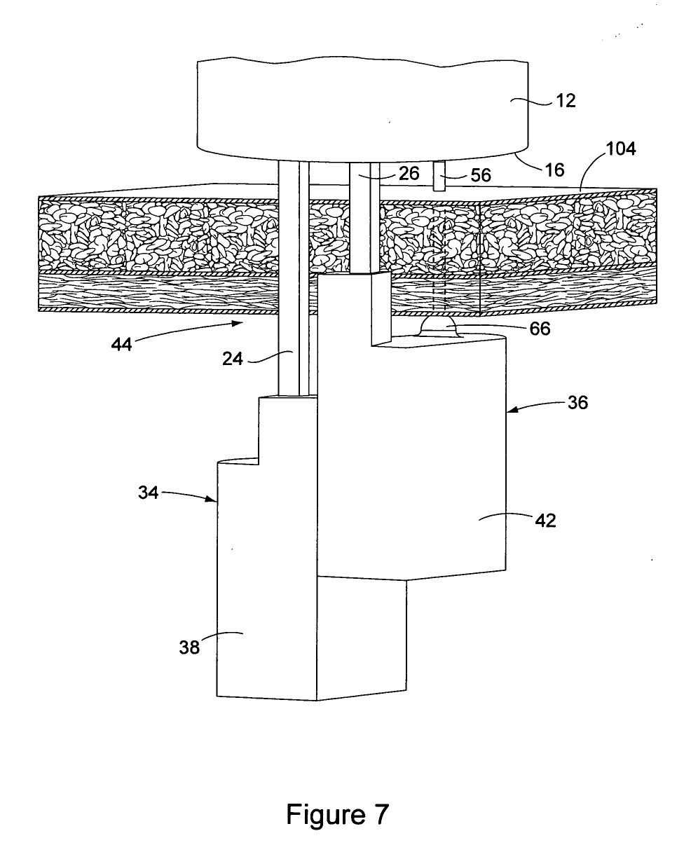

The instrument 10 is then rotated in the incision approximately 180 degrees. This positions the needle holders 34, 36 on the opposite side of the incision, with the second portion of the body tissue 104 on the opposite side of the incision in the spacing 44 between the needle holders 34, 36 and the handle distal end 16. This positioning of the instrument 10 is shown schematically in FIG. 6. The trigger 32 for the second needle holder 36 is then pulled by the surgeon toward the handle distal end 14 in a repeat of the procedure previously described. This causes the second needle holder 36 to move toward the handle distal end 16, and thereby causes the second needle 56 to penetrate through the second tissue portion 104 and into the second needle receiver 94 as shown in FIG. 7. The second trigger 32 is then released, causing the second needle holder 36 to move back to its first, original position in the same manner as previously described for the first needle holder 34. This causes the second needle receiver 94 to firmly hold and remove the second needle 56 from the second needle holder 36 as the second needle holder 36 moves back toward its first position relative to the handle 12.

The above procedure results in the pair of needles 54, 56 being passed through the two tissue portions 102, 104 on opposite sides of the incision, with the length of suture 82 connecting the two needles 54, 56 extending across the incision. Withdrawing the instrument 10 from the incision with the two needles 54, 56 held by the respective needle receivers 92, 94 causes the respective ends 84, 86 of the length of suture to be pulled through the needle holes at the opposite sides of the incision. Removing the instrument 10 from the incision provides access to the opposite ends 84, 86 of the suture held by the needle receivers 92, 94. Tying and tightening a knot in the suture ends then closes the incision.

The body tissue incision closing instrument and its method of operation described above provides a quick and effective means for closing incisions deep in body tissue. Although only a single embodiment of the instrument has been described, it should be understood that modifications and variations may be made to the instrument and its method of use without departing from the intended scope of the following claims.

1. A body tissue incision closing instrument comprising:
a handle having a length with opposite proximal and distal ends, the handle distal end being configured for insertion into a body tissue incision and the handle proximal end being configured for manual manipulation by a person's hand;
a needle holder on the handle distal end, the needle holder being movable between first and second positions of the needle holder relative to the handle distal end;
a needle removably mounted on the needle holder for movement of the needle with the needle holder; and,
a needle receiver on the handle at a position where the needle receiver receives the needle from the needle holder in response to the needle holder moving from the first position to the second position, and the needle receiver being operative to remove the needle from the needle holder in response to the needle holder moving from the second position to the first position.

2. The instrument of claim 1, further comprising:
a length of suture secured to the needle.

3. The instrument of claim 1, further comprising:
the needle having a point at one end and a length of suture secured to an opposite end of the needle, and the needle point being directed at the handle distal end.

4. The instrument of claim 1, further comprising:
a spacing between the needle and the needle receiver when the needle is in the first position, and the needle moving across the spacing when the needle is moved from the first position to the second position.

5. The instrument of claim 1, further comprising:
a rod mounted on the handle for reciprocating movement of the rod along the handle length, the rod being operatively connected to the needle holder for moving the needle holder between the first and second positions in response to reciprocating movement of the rod.

6. The instrument of claim 5, further comprising:
a trigger operatively connected to the rod, the trigger being positioned adjacent the handle proximal end where the trigger is engagable by a finger of the person's hand manipulating the handle proximal end.

7. The instrument of claim 1, further comprising:
the needle holder being a first needle holder of first and second needle holders on the handle distal end, the first and second needle holders being movable between first and second positions of the needle holders relative to the handle distal end; and,
the needle being a first needle of first and second needles removably mounted on the respective first and second needle holders.

8. The instrument of claim 7, further comprising:
the first and second needle holders being positioned side-by-side and being independently movable.

9. The instrument of claim 7, further comprising:
a length of suture having opposite first and second ends, the suture first end being secured to the first needle and the suture second end being secured to the second needle.

10. The instrument of claim 7, further comprising:
first and second rods mounted on the handle for reciprocating movement of the rods along the handle length, the first and second rods being operatively connected to the respective first and second needle holders for moving the needle holders in response to moving the rods.

11. The instrument of claim 10, further comprising:
first and second triggers operatively connected to the respective first and second rods, the first and second triggers being positioned adjacent the handle proximal end where the first and second triggers are engagable by a finger of the person's hand manipulating the handle proximal end.

12. A body tissue incision closing instrument comprising:
a handle having a length with opposite proximal and distal ends, the handle distal end being configured for insertion into a body tissue incision and the handle proximal end being configured for manual manipulation by a person's hand;
an actuator mounted on the handle for movement along the handle length between first and second positions of the actuator relative to the handle; and,
a needle operatively connected to the actuator with a point of the needle directed toward the handle distal end, the needle being movable with the actuator toward the handle distal end in response to the actuator moving from the first position to the second position.

13. The instrument of claim 12, further comprising:
a length of suture connected to the needle.

14. The instrument of claim 13, further comprising:
a spacing between the needle point and the handle distal end when the actuator is in the first position, and the needle point being movable across the spacing in response to the actuator moving from the first position to the second position.

15. The instrument of claim 14, further comprising:
the needle being removably connected to the actuator.

16. The instrument of claim 12, further comprising:
a trigger operatively connected to the needle by the actuator, the trigger being positioned on the handle adjacent the handle proximal end where the trigger is engagable by a finger of the person's hand manipulating the handle proximal end.

17. The instrument of claim 12, further comprising:
the actuator being a first actuator of first and second actuators mounted on the handle for independent movement between first and second positions of the actuators relative to the handle; and,
the needle being a first needle of first and second needles operatively connected to the first and second actuators and positioned side-by-side with points of the first and second needles directed toward the handle distal end, the first and second needles being movable with the first and second actuators.

18. The instrument of claim 17, further comprising:
a length of suture having opposite ends secured to the first and second needles.

19. A body tissue incision closing instrument comprising:
a handle having a length with opposite proximal and distal ends, the handle distal end being configured for insertion into a body tissue incision and the handle proximal end being configured for manual manipulation by a person's hand;
a means for holding a needle at the handle distal end;
a means for moving the means for holding a needle between first and second positions of the means for holding a needle relative to the handle distal end;
a needle removably held by the means for holding a needle; and,
a means for receiving the needle held by the means for holding a needle and for removing the needle from the means for holding a needle.

20. The instrument of claim 19, further comprising:
a length of suture secured to the needle.

21. The instrument of claim 19, further comprising:
the needle having a point at one end and a length of suture secured to an opposite end of the needle; and,
the means for holding the needle directing the needle point at the means for receiving the needle with a spacing between the needle point and the means for receiving the needle.

22. The instrument of claim 19, further comprising:
the means for holding a needle being one of first and second separate means for holding a needle at the handle distal end;
the means for moving the means for holding a needle being one of first and second separate means for moving the respective first and second means for holding a needle between first and second positions of the respective first and second means for holding a needle relative to the handle distal end; and,
the needle being one of first and second needles removably held by the respective first and second means for holding a needle.

23. The instrument of claim 22, further comprising:
a length of suture having opposite first and second ends secured to the respective first and second needles.

24. A method of closing an opening in body tissue, the method comprising:
providing a handle with a length with opposite proximal and distal ends;
removably mounting first and second needles on the handle adjacent the handle distal end;
securing a length of suture to the first and second needles, with the length of suture having opposite ends secured to the first and second needles;
positioning the handle distal end adjacent the opening in the body tissue;
deploying the first needle from the handle distal end through the body tissue at one side of the opening;
deploying the second needle from the handle distal end through the body tissue at a second side of the opening;
retrieving the deployed first and second needles and the attached length of suture through the opening; and,
binding together the suture ends secured to the first and second needles to close the opening.

25. The method of claim 24, further comprising:
providing a needle receiver on the handle distal end for receiving the deployed first and second needles.

26. The method of claim 24, further comprising:
positioning the handle distal end in the tissue opening prior to deploying the first and second needles.

27. The method of claim 26, further comprising:
keeping the handle distal end in the tissue opening while deploying the first and second needles, and while retrieving the first and second needles.

28. The method of claim 24, further comprising:
positioning the first and second needles on the handle distal end with points of the needles directed toward the handle distal end.
